(19) United States
(12) Patent Application Publication
(10) Pub. No.: US 2010/0076303 A1
(43) Pub. Date: Mar. 25, 2010

(54) Lateral Deployment Catheter

(75) Inventor: David J. McKinley, Chanhassen, MN (US)

Correspondence Address: INSKEEP INTELLECTUAL PROPERTY GROUP, INC, 2281 W. 190TH STREET, SUITE 200, TORRANCE, CA, 90504, US

(73) Assignee: superDimension, Ltd.

(21) Appl. No.: 12/564,833

(22) Filed: Sep. 22, 2009

Related U.S. Application Data
(60) Provisional application No. 61/099,125, filed on Sep. 22, 2008.

(51) Int. Cl. A61B 5/055; A61B 10/02; A61B 18/02; A61B 18/18

(52) U.S. Cl. 600/424; 600/562; 606/21; 606/41

(57) ABSTRACT

A probe or catheter, usable with a navigation system, includes a longitudinal lumen that curves outwardly through a sidewall prior to reaching a distal tip of the probe. The distal tip contains a position sensor as part of the navigation system. The lumen provides a working channel that guides a tool into a sidewall of a branched structure without requiring the distal end of the probe to be facing the branched structure.

### BACKGROUND OF THE INVENTION

Breakthrough technology has emerged which allows the navigation of a catheter tip through a tortuous channel, such as those found in the pulmonary system, to a predetermined target. This technology compares the real-time movement of a sensor against a three-dimensional digital map of the targeted area of the body (for purposes of explanation, the pulmonary airways of the lungs will be used hereinafter, though one skilled in the art will realize the present invention could be used in any body cavity or system: circulatory, digestive, pulmonary, to name a few).

Such technology is described in U.S. Pat. Nos. 6,188,355; 6,226,543; 6,558,333; 6,574,498; 6,593,884; 6,615,155; 6,702,780; 6,711,429; 6,833,814; 6,974,788; and 6,996,430, all to Gilboa or Gilboa et al.; and U.S. Published Applications Pub. Nos. 2002/0193686; 2003/0074011; 2003/0216639; 2004/0249267 to either Gilboa or Gilboa et al. All of these references are incorporated herein in their entireties.

The systems in use that use this technology employ a steerable guide or probe that has a sensor on or near its distal end. Once the probe has been navigated, using the system, to a target location, a catheter or sheath, also referred to as an extended working channel (hereinafter “EWC”), is advanced over the probe until the distal end of the EWC reaches the distal end of the probe. The probe may then be retracted, and the EWC is used as a conduit through which tools are advanced to the target site.

Typically, the target site is a lesion. Lesions in the lungs are often found to the side of an airway. Thus, in order to advance a tool to the target, the distal end of the EWC must be angled toward the lesion. Establishing this angle is easy using the probe because the probe includes the sensor, which has six degrees of freedom. However, once the probe is removed, the positional information regarding the distal end of the EWC is gone. At this point it is not only difficult to keep the tip of the EWC at the desired angle, there is no information provided to the user as to whether the angle is being maintained or if the distal tip of the EWC has moved.

Furthermore, there is no way to track where the tip of a tool advanced through the EWC is, or where a tool has been. Certain procedures, such as marker placement or multiple biopsy sampling, require that a tool be advanced into a lesion multiple times. It would be desirable to have information regarding both the present location of the tool tip and historical data regarding where the tip has been.

Hence, there is a need for a device that utilizes the aforementioned sensor technology to deploy a tool into a lateral target while providing real-time as well as historical tool position data.

### SUMMARY OF THE INVENTION

One aspect of the present invention relates to a probe device that has a sensor incorporated into its distal tip. Proximal of the sensor is a lateral port leading to a central lumen that extends proximally through the device and can be used to advance a tool therethrough. The port is curved laterally, such that a tool advanced through the lumen will exit the probe's distal portion at a predictable angle to the probe's longitudinal axis. Hence, the tool may be directed to a lateral lesion without bending the probe.

Another aspect of the present invention is to provide a system that provides real-time as well as historical tool position data. By tracking the relative position of the tool within the probe, and by simultaneously tracking the position and orientation of the probe sensor, the position and orientation of the tool can be estimated with excellent accuracy. By recording the various tool positions over time, a display may be provided that shows where the tool has been. Furthermore, if markers are being placed, the positions of the various markers may be displayed such that it can be determined where further markers should be placed.

Similarly, another aspect of the present invention provides a biopsy tool deployed through the aforementioned lateral port of the probe. The biopsy tool is used to take multiple samples. Using the historical recording and displaying capabilities of the present invention, the display may altered to not only show where biopsy samples have been taken, but also the tissue that has been removed.

Because the tool is deployed laterally from an axially-aligned probe, the present invention may be used to acquire circumferential samples by deploying a scraping or brush-like device and then rotating the probe within the airway.

### BRIEF DESCRIPTION OF THE DRAWINGS

FIG. 1 is a perspective view of a distal tip of an embodiment of a probe of the present invention;

FIG. 2 is a perspective view of a distal tip of an embodiment of a probe of the present invention with a tool deployed therefrom;

FIG. 3 is a perspective view of a proximal end of an embodiment of the present invention; and,

FIG. 4 is a depiction of a display utilizing data made available by the present invention.

### DETAILED DESCRIPTION OF THE INVENTION

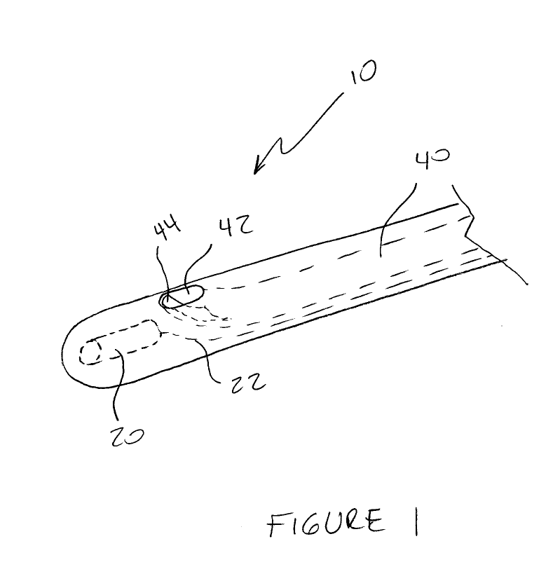

Referring now to the figures, and first to FIG. 1, there is shown a distal end of a probe 10 of the present invention. The probe, or catheter, 10 generally includes a sensor 20 and defines a lumen 40. The sensor 20 can be any active or passive sensor component of a navigation or position sensor. Examples of some sensors are described in the aforementioned incorporated references.

Preferably, the sensor 20 senses position and orientation and provides to a probe navigation system, data pertaining to six degrees of freedom, specifically, positional data in three orthogonal axes, as well as orientation data pertaining to pitch, roll, and yaw. The sensor 20 may be wireless or may be connected to a controller via a wire bundle 22.

The lumen 40 is formed within the probe 10 and extends longitudinally from a proximal end of the probe 10 to a point proximal of the sensor 20. Prior to reaching the sensor 20, the lumen curves laterally and forms a lateral port 42. Hence, the sensor 20 is encased within a solid segment of catheter material.

Because the probe 10 is intended to be used with a variety of tools, including needles and various cutting tools, it is preferable to harden the material at the distal curvature of the lumen. Alternatively, as shown in FIG. 1, an inset 44 may be provided to prevent a needle or other tool from penetrating the catheter material rather than following the curve to the lateral port 42. Other tools useable with the probe 10 include various biopsy tools, cryo tools, ablation tools, radiation tools or any other tools useable to treat tissue.

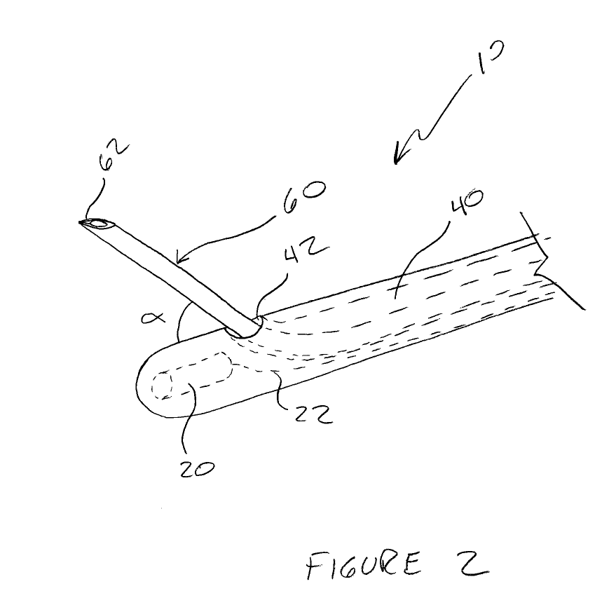

FIG. 2 shows a tool 60, such as a needle, deployed through the lateral port 42. For a given tool 60, the deployment angle a is relatively constant. Thus, knowing the relationship between the tool 60 and the probe 10 allows the position of the tip 62 of the tool 60 to be calculated. Because the actual position and orientation of the sensor 20 is known using the navigational system, the actual position of the tool tip 62 may also be calculated and displayed.

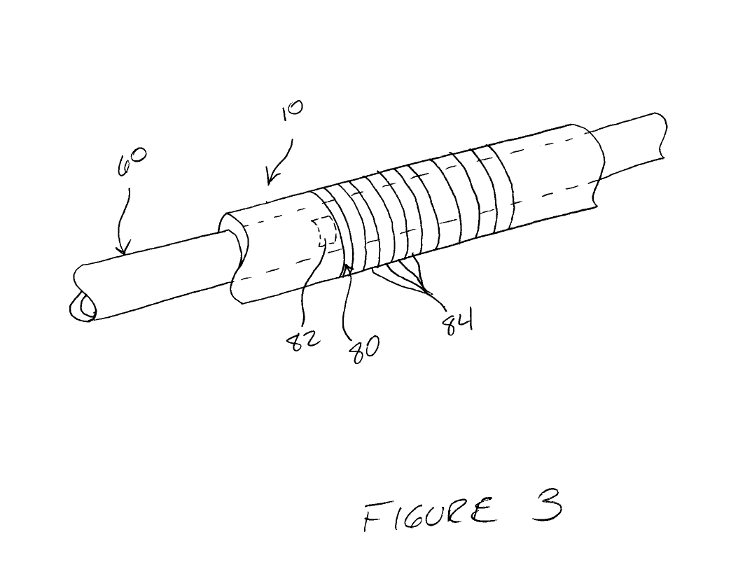

FIG. 3 shows an example of a device 80 that can be used to monitor the position of the tool 60 relative to the probe 10. The device 80 includes a tool sensor 82 and a probe sensor 84. The embodiment shown in FIG. 3 includes a single tool sensor 82 and a plurality of probe sensors 84. As the tool sensor 82 passes the probe sensors 84, the probe sensors 84 are able to detect the tool sensor 82. Hence, the position of the tool 60 is known. Alternatively, a plurality of tool sensors 82 along the tool 60 could be used in combination with one or more probe sensors 84. One skilled in the art will realize that there are many known proximity sensor designs that would work well for this application.

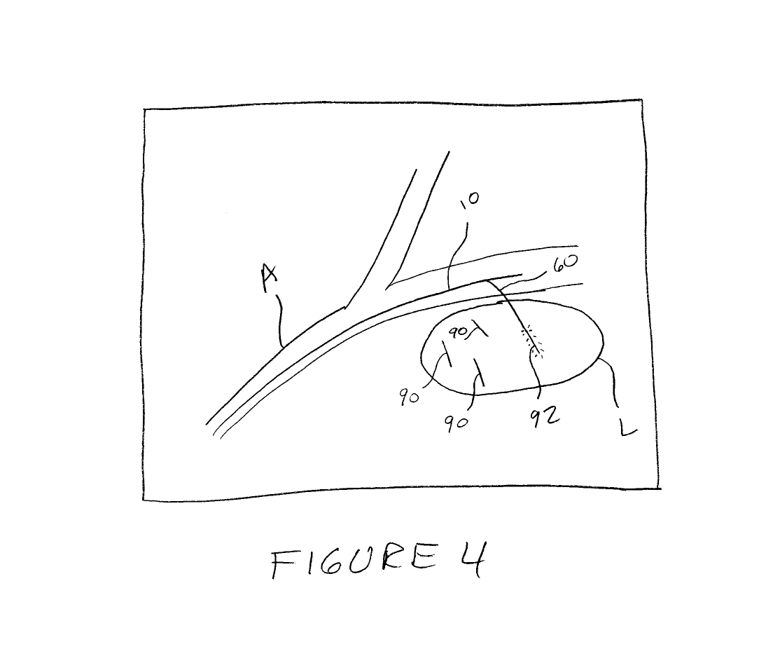

FIG. 4 shows an example of a display 100 utilizing the data made available by the present invention. The display 100 shows a segment of a targeted airway A and a targeted lesion L. In this scenario, the tool 60 extending from probe 10 is a marker placement tool being used to place markers 90 and 92 into the lesion L. The display includes indications of markers 90 that have already been placed in the lesion L as well as a visualization of the position of the probe 10, the tool 60 and the marker 92 being placed. It may be advantageous to somehow highlight the marker 92 to distinguish it from those markers 90 that have already been placed.

Though not shown in the Figures, the probe 10 may include a steering mechanism, such as one of the steering mechanisms disclosed in the aforementioned incorporate references.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A probe for accessing tissue in a branched structure comprising:
a catheter having a lumen and a distal end, said central lumen extending longitudinally through said catheter and curving laterally through a sidewall of said catheter to form a lateral port proximal of said distal end;
a sensor located in said distal end of said catheter, distal of said lateral port, operatively associated with a navigation system to provide location information thereto such that positions of said distal end may be displayed and recorded.

2. The probe of claim 1 further comprising a flexible tool, advanceable through said lumen and flexible enough to pass through said lateral port without changing an orientation of said catheter distal end.

3. The probe of claim 2 wherein said flexible tool comprises a biopsy tool.

4. The probe of claim 2 wherein said flexible tool comprises a marker placement tool.

5. The probe of claim 1 wherein said sensor comprises a passive sensor.

6. The probe of claim 1 wherein said sensor comprises an active sensor.

7. The probe of claim 1 wherein said sensor provides information pertaining to six degrees of freedom.

8. The probe of claim 1 further including a reinforcing mechanism at said distal end that prevents a tool from penetrating material of said catheter while following a curve of said lumen.

9. The probe of claim 2 further comprising a tool position mechanism capable of determining a position of a distal end of said tool relative to said catheter such that said tool position may be displayed and recorded.

10. The probe of claim 9 wherein said tool position mechanism comprises a plurality of probe sensors along said catheter and at least one tool sensor disposed on said tool such that as said tool sensor passes one of said plurality of probe sensors, a position of said tool relative to said catheter is known.

11. The probe of claim 9 wherein said tool position mechanism comprises a plurality of tool sensors along said tool and at least one probe sensor disposed on said catheter such that as said tool sensors pass said at least one probe sensor, a position of said tool relative to said catheter is known.

12. A method of treating lung tissue comprising:
navigating a probe to a target site such that a distal end of said probe is just distal of said target site;
extending a tool through a sidewall of said probe to said target site;
treating said tissue using said tool.

13. The method of claim 12 wherein treating said tissue using said tool comprises biopsying said tissue.

14. The method of claim 12 wherein treating said tissue comprising freezing said tissue.

15. The method of claim 12 wherein treating said tissue comprises radiating said tissue.

16. The method of claim 12 wherein treating said tissue comprises ablating said tissue.

17. The method of claim 12 wherein navigating a probe to a target site such that a distal end of said target site is just distal of said target site comprises:
receiving location information from a sensor located in said distal end of said probe;
displaying a representation of a location of said probe relative to an image of the lungs using said location information.

18. The method of claim 17 wherein receiving location information from a sensor located in said distal end of said probe comprises receiving location and orientation information.

19. The method of claim 12 further comprising:
calculating a position of said tool in the lungs;
recording said position; and,
displaying a representation of said position.

20. A probe for use with an electromagnetic probe navigation system comprising:
a catheter having a longitudinal lumen that terminates through a sidewall thereof prior to reaching a distal end of said catheter;
a position sensor in said distal end of said catheter capable of communicating position information to said electromagnetic probe navigation system;
a tool extendable through said longitudinal lumen and beyond said sidewall.

21. The probe of claim 20 wherein said tool comprises a flexible biopsy needle.
